(19) United States
(12) Patent Application Publication
(10) Pub. No.: US 2026/0364260 A1
(43) Pub. Date: Nov. 19, 2026

(54) NEEDLE HEAD FOR AN INJECTION DEVICE FOR A MEDICAL ACTIVE SUBSTANCE, AND INJECTION DEVICE HAVING SUCH A NEEDLE HEAD

(71) Applicant: INDUCTIO AG, Hergiswil (CH)

(72) Inventor: Horst KOLLER, Uznach (CH)

(73) Assignee: INDUCTIO AG, Hergiswil (CH)

(21) Appl. No.: 19/138,578

(22) PCT Filed: Dec. 13, 2023
(86) PCT No.: PCT/EP2023/085626

§ 371 Date: Jun. 12, 2025

(30) Foreign Application Priority Data
Dec. 14, 2022 (EP) 22213561.8

(51) Int. Cl. A61M 5/24; A61M 5/20; A61M 5/32

(52) U.S. Cl. CPC A61M 5/24; A61M 5/20; A61M 5/321; A61M 2005/2444

(57) ABSTRACT

A needle head for an injection device for a medical active substance, in particular a pen injector, is intended to particularly facilitate the filling of even large numbers of ampoules for such injectors. The needle head comprises an inner cap which can be slid onto a connecting piece of an active substance ampoule and which comprises a number of latching elements in a region designed as a bulging cap, and an outer cap which can be slid onto the inner cap and which, when slid onto the inner cap, fixes the latching elements radially.

### CROSS-REFERENCE TO RELATED APPLICATION

This application is a national phase of International Application No. PCT/EP2023/085626, filed Dec. 13, 2023, which is hereby incorporated by reference in its entirety.

### FIELD

This disclosure relates to a needle head for an injection device for a medical agent, in particular for a so-called pen injector. It also relates to an injection device with such a needle head.

### BACKGROUND

A large number of suitable injection devices are provided for patients with chronic diseases who need to be administered medical agents on a regular basis and who, in particular, should also be able to administer these injections themselves. In particular, it should also be possible to administer several injections preferably from the contents of a storage container contained in the injection device by means of such an injection device. Such injection devices are used, for example, by diabetics to give themselves regular injections of insulin. Such injection devices for self-use are usually also referred to as “pen injectors”, particularly as they are usually similar in shape and size to conventional pens in terms of their suitability for everyday use.

Such a pen injector, or the injection device in general, usually comprises a so-called ampoule as the actual active substance container, in which the active substance, for example the insulin to be administered, is suitably stored. Such an ampoule is provided prefilled and, for actual use, is provided with the injection needle on a needle head prepared for the attachment of an injection needle on the one hand and with a suitable actuating element on its end opposite the needle head on the other. The actuating element acts on a stopper slidably arranged in the ampoule, via which the active ingredient can be supplied to the injection needle attached to the needle head and administered via this.

Particularly in the case of diabetes and when administering insulin, the dose to be injected usually has to be adjusted to the patient's current blood glucose level, usually even immediately before the respective injection. For this purpose, the injection device can be designed to deliver an adjustable dose, whereby such an adjustment is usually made via the correspondingly designed actuating element. This can, for example, have a piston rod with an external thread, which can be axially displaceable in the housing of the injection device and thus in the ampoule. The dose can then be set, for example, by turning an outer housing part with an internal thread, which moves the piston rod axially by a predetermined distance. This enables comparatively precise dosing of the amount of liquid to be injected.

Such injection devices are now widely used. As mentioned above, the active ingredient is provided to the user in pre-filled ampoules. Until they are used, these ampoules should therefore be securely and tightly sealed to ensure good shelf life and easy handling in order to minimize or, if possible, completely avoid any unwanted loss of active ingredient. Furthermore, such ampoules should be particularly easy to produce in large quantities in view of the high demand and should also be fillable on an industrial scale.

### SUMMARY

This disclosure is therefore based on the task of providing a needle head intended in particular for use with the ampoule of such a pen injector, with which the filling of even large numbers of such ampoules is particularly facilitated. Furthermore, an injection device with such a needle head is to be specified.

With regard to the needle head, this task is performed according to this disclosure with an inner cap which can be slid onto a connection piece of an active substance ampoule and which comprises a number of latching elements in an area designed as a bulging cap, and with an outer cap which can be slid onto the inner cap and which, when slid onto the inner cap, fixes the latching elements radially.

This disclosure is based on the consideration that, in order to simplify the filling process, the sealing of the ampoule after the actual filling of the active ingredient into the ampoule should be particularly simple and efficient. For this purpose, in accordance with one aspect of this disclosure and in an embodiment which is regarded as independently inventive, the needle head and the ampoule adapted thereto are designed for a novel filling concept, in which the filling of the active ingredient into the ampoule is to take place via the distal end of the ampoule, which is provided with the needle head for attaching the injection needle. For this purpose, the needle head should be designed for simple and easily controllable attachment to the distal end of the ampoule after it has been filled with the active ingredient. According to one aspect of this disclosure, the needle head is designed for attachment to the ampoule via a snap-on or snap-in connection, in which the needle head is pushed or pressed onto a corresponding connection piece of the ampoule in an essentially linear movement. However, in order to ensure a reliable hold of the needle head on the ampoule on the one hand and a high degree of tightness when closing the ampoule on the other, the needle head is designed in several parts in accordance with one aspect of this disclosure. In particular, it should have an inner cap as the first component, which can be attached to the connection piece of the ampoule in a comparatively simple manner and can be latched to it and also has a suitable sealing element for sealingly closing the distal end of the ampoule. As a second component, it should also have a suitable securing mechanism with which the inner cap attached to the connecting piece can be easily fixed in position.

Advantageous embodiments of this disclosure are the subject of the subclaims.

Advantageously, the inner cap of the needle head comprises a sealing body made of a softer material, preferably TPE, compared to the bulging cap.

In a particularly advantageous embodiment, the outer cap of the needle head is provided with an external thread on its outer casing, via which in particular a needle attachment carrying the injection needle can be attached. Preferably, the external thread is thus adapted to the intended needle attachment, for example a standard needle attachment for pen injectors, with regard to its thread or geometry parameters. In the assembled state, i.e., completely pushed onto the connection piece of the ampoule, the area of the outer shell of the outer cap provided with the external thread covers the part of the inner cap designed as a bump cap with latching elements and surrounds it. According to one aspect of this disclosure, the external thread thus contributes in an additional stabilizing manner to the radial fixation of the Ras elements of the inner cap and thus to its fixation and secure positioning on the connection piece of the ampoule.

With respect to the injection device with an active ingredient ampoule, the above task is solved in accordance with one aspect of this disclosure in that the active ingredient ampoule is sealed at its distal end with the sealing body of a needle head of the type described above.

According to one aspect of this disclosure, the advantages of the described design of the needle head can be used particularly well for prefilled active ingredient ampoules, especially for closing the active ingredient ampoules after they have been filled with active ingredient. Advantageously, the active ingredient ampoule of the injection device is thus prefilled with the active ingredient to be administered.

According to a further aspect of this disclosure, the injection device is designed as a pen injector.

Further advantageous and/or independently inventive aspects of this disclosure are indicated in the sub-claims and/or explained in more detail in the following embodiment example.

### BRIEF DESCRIPTION OF THE DRAWINGS

An embodiment of this disclosure is explained in more detail with reference to a drawing. It shows:

FIG. 1 is a lateral view of an injection device;

FIG. 2 illustrates the injection device according to FIG. 1 in longitudinal section;

FIG. 3 illustrates an ampoule of the injection device according to FIG. 1 in perspective view in exploded view;

FIG. 4 illustrates an ampoule of the injection device according to FIG. 1 in longitudinal section in exploded view;

FIG. 5 illustrates a stopper guided in the ampoule according to FIG. 3 in longitudinal section;

FIG. 6 is a perspective view of the stopper according to FIG. 5;

FIG. 7 illustrates the plug according to FIG. 5 in lateral view;

FIG. 8 is a perspective view of the plug according to FIG. 5;

FIG. 9 illustrates a longitudinal section of a needle head intended for attachment to the distal end of the ampoule according to FIG. 3;

FIG. 10 is a lateral view of the needle head according to FIG. 9;

FIG. 11 illustrates a longitudinal section of the needle head attached to the distal end of the ampoule according to FIGS. 3, 4;

FIG. 12 is a lateral view of the needle head attached to the distal end of the ampoule according to FIGS. 3, 4;

FIG. 13 illustrates an alternative embodiment of a needle head intended for attachment to the distal end of the ampoule according to FIG. 3 in longitudinal section;

FIG. 14 is a lateral view of the needle head according to FIG. 13;

FIG. 15 illustrates a longitudinal section of the alternative needle head attached to the distal end of the ampoule as shown in FIGS. 3, 4 as shown in FIG. 13;

FIG. 16 is a lateral view of the needle head attached to the distal end of the ampoule as shown in FIGS. 3, 4 as shown in FIG. 13;

FIG. 17 illustrates a needle attachment for mounting on a needle head as shown in FIGS. 9, 13 in perspective section;

FIG. 18 illustrates the distal end of the ampoule according to FIGS. 3, 4 after removal of the sealing plate;

FIG. 19 illustrates the distal end of the ampoule according to FIGS. 3, 4 with screwed-on needle holder in longitudinal section; and

FIG. 20 is a sequence of steps in the use of the injection device according to FIG. 1, each in longitudinal section.

Identical parts are marked with the same reference signs in all figures.

### DETAILED DESCRIPTION

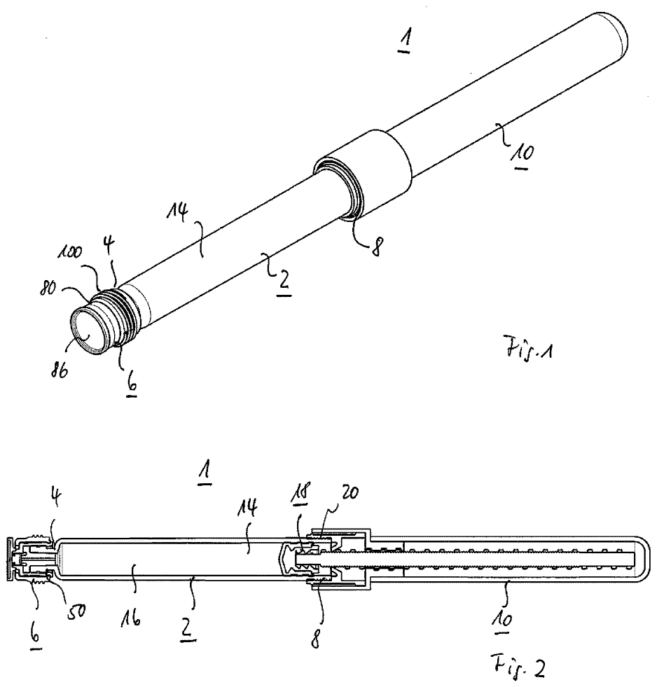

The injection device 1 according to FIG. 1 is designed as a so-called pen injector and is intended for multiple dosed delivery of a medical active ingredient, in particular insulin or the like. It essentially comprises three components, namely an ampoule 2 (also referred to as a cartridge or ampoule body) forming an active ingredient container, which carries a needle head 6 at its distal end 4 and is connected to an actuating element 10 at its proximal end 8. In the overall view in FIG. 1 and also in the representation in longitudinal section in FIG. 2, the injection device 1 is shown in each case with the injection needle not yet attached to the needle head 6 and thus in a transport or storage state or also in a ready-for-sale state. In accordance with a conventional design, the ampoule 2 comprises a cylindrical or tubular hollow body 14 forming a syringe housing, which is intended to hold the active medical ingredient, for example insulin. In this hollow body 14, a stopper 18 is guided within the syringe channel 16 forming the actual active ingredient chamber, which closes the interior of the hollow body 14 to its proximal end 8 in a sealing manner. In addition, the needle head 6 attached to the distal end 4 of the hollow body 14 in the state shown, i.e., still without the injection needle, is designed in such a way that it seals the interior of the hollow body 14. The ampoule 2 is thus designed in its entirety as a sealed container in which the active medical ingredient can be stored and kept for a certain storage period. In particular, the ampoule 2 can thus be designed as a pre-filled active ingredient container.

With regard to the choice of material for the ampoule 2 or the hollow body 14 forming it, which is considered to be inventive in its own right, particular account is taken of high demands on the reliable temporary storage of the active substance, in particular insulin, together with a particularly high level of safety when handling the components. The cylindrical hollow body 14 can be made of COC, PC or PP, but in the embodiment shown it is made of the high-performance plastic cyclo-olefin polymer (COP), which is considered to be independently inventive. This material is characterized by its high breaking strength and glass-like transparency. It also does not release alkali ions, so that the risk of a pH shift in the active ingredient is eliminated.

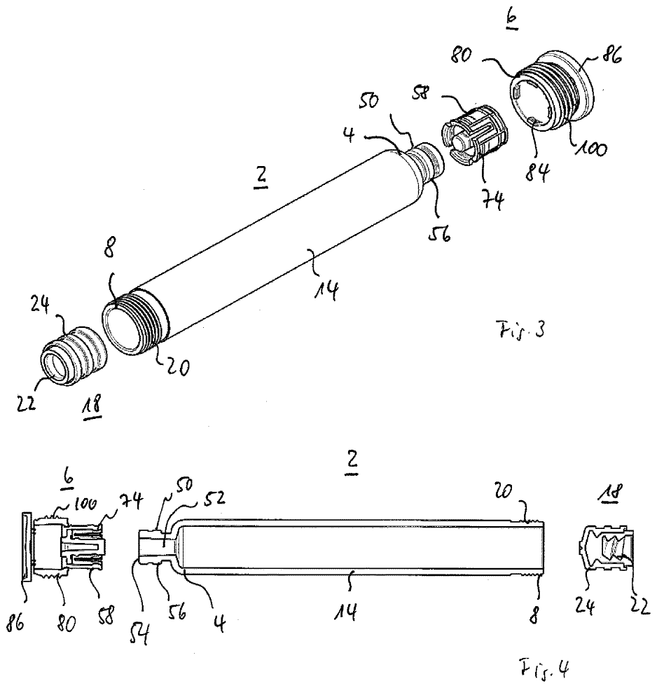

The ampoule 2, which is regarded as independently inventive, is shown with its closure elements on both sides, namely the needle head 6 on the one hand and the stopper 18 on the other hand, in FIG. 3 in perspective view and in FIG. 4 in longitudinal section, in each case in exploded view. The hollow body 14 is provided with an external thread 20 at its proximal end 8 in order to enable the connection with the actuating element 10. The plug 18 can be inserted into the proximal end 8, which in the inserted state closes the hollow body 14 sealingly towards its proximal end 8.

The stopper 18 is displaceable within the hollow body 14 via the actuating element 10 arranged at the proximal end of the hollow body 14, so that, for example, the active substance held in the hollow body 14 can be discharged via a hollow needle mounted in the needle head 6 and thus applied. The stopper 18 is dimensioned in such a way that it lies tightly against the inner wall of the hollow body 14; the reservoir for the active ingredient is thus formed in the longitudinal region of the interior of the hollow body 14 between the distal end 4 and the stopper 18, in which the active ingredient can be stored. When the hollow needle is attached to the needle head 6, its proximal end protrudes into this reservoir volume, whereby the stopper 18 can be displaced within the hollow body 14 in the direction of the distal end 4, so that the active ingredient is pressed out of the interior by the hollow needle. In particular, this can be in a silicone-free configuration.

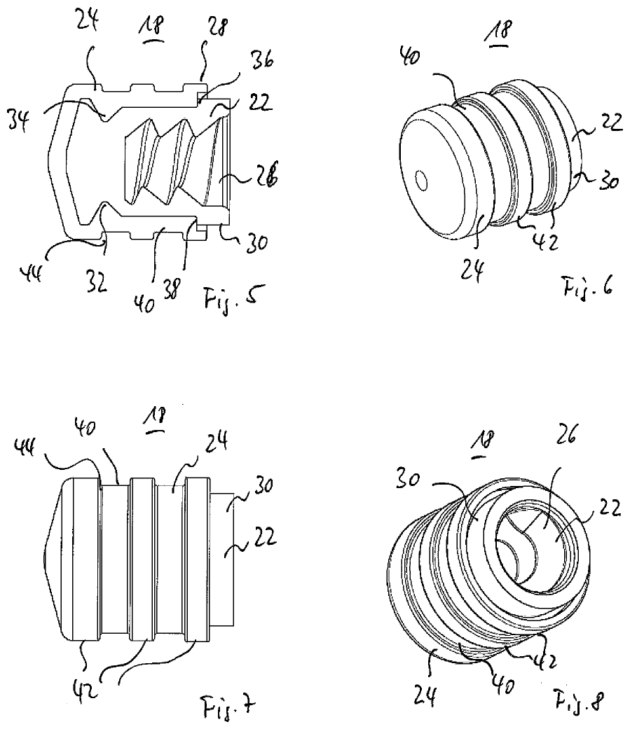

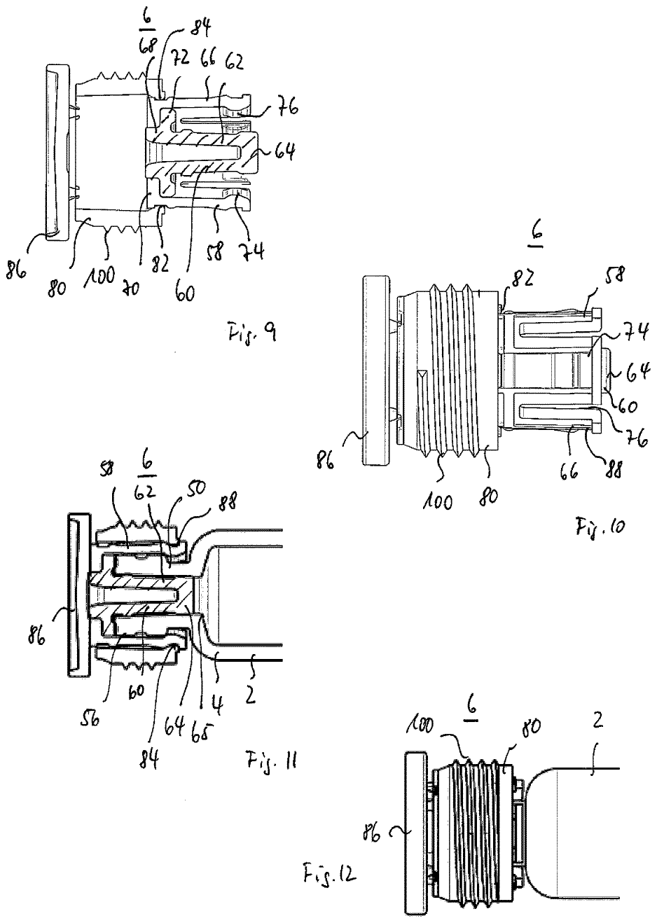

In order to satisfy even the highest demands with regard to tightness on the one hand and sliding properties within the hollow body 14 on the other, the stopper 18 is designed to be multi-component, in particular two-component, in an embodiment which is regarded as independently inventive. Details of the design of the stopper 18 can be seen in the illustrations in longitudinal section in FIG. 5, in perspective view in FIG. 6, in lateral view in FIG. 7 and in further perspective view “from behind” in FIG. 8.

The two-component or “2K plug” according to one aspect of this disclosure comprises a central plug body 22 made of a comparatively hard plastic, in the embodiment example and according to one aspect of this disclosure made of polypropylene (PP), which is surrounded by a sealing jacket 24 formed of a comparatively softer plastic material, in the embodiment example and according to one aspect of this disclosure TPE. The central plug body 22 essentially serves to provide dimensional and structural stability and to transmit the forces introduced, in particular in the longitudinal direction when the plug 18 is displaced within the hollow body 14. For this purpose, the central plug body 22 can be provided on the inside with a receiving channel 26, for example with a thread, via which a connection to the actuating element 10 is made possible. Due to the design of the stopper 18 as a 2-component stopper with a combination of the central stopper body 22 with the comparatively soft sealing jacket 24, which is regarded as independently inventive, the contact pressure of the sealing jacket 24 on the inner wall of the hollow body 14 and thus the tightness of the system can be adjusted just as well and precisely as the sliding friction forces when the stopper 18 is displaced in the hollow body 14 by virtue of a suitable choice of geometry and parameters (for example, precise material composition or selection of the plastic used) of these components. This means that a stopper 18 constructed in this way can be specifically adapted to the requirements when using the injection device 1, for example for a highly precisely dosed delivery of the active ingredient by the patient himself with correspondingly sensitive handling.

Furthermore, the combination of these components can prevent the shrinkage that usually occurs when using pure TPE stoppers after a long storage period and thus a loss of tightness. A 2-component stopper of this type therefore also reliably enables comparatively long storage times for prefilled syringes.

This method of construction provided according to one aspect of this disclosure, i.e., the provision of the inner, comparatively harder stopper body 22 with a sheathing by the comparatively softer sealing jacket 24, with the stable thread provided in the receiving channel 26 is considered to be particularly advantageous for use in an injection device 1 designed as a pen injector. Thanks to this design, the stopper body 22 and with it the stopper 18 as a whole can be firmly connected to an actuating element of the injection device 1. As a result, axial displacements of the stopper 18 within the hollow body 14 can take place both in the “forward” and in the “backward” direction, i.e., selective and, if required, pushing or also pulling effects can be exerted on the stopper 18.

According to one aspect of this disclosure, the sealing jacket 24 almost completely surrounds the central plug body 22 as viewed in the longitudinal direction, with a flange 30 at the end remaining uncovered by the sealing jacket 24 only in the proximal connection region 28 of the plug body 22. The central plug body 22 is provided with a circumferential retaining groove 32 for positive fixing of the sealing jacket 24, into which an inner lip 34 of the sealing jacket 24 engages. Furthermore, the central plug body 22 has a circumferential edge 36 in the area of the end flange 30, on which a corresponding inner edge 38 of the sealing jacket 24 rests.

In one embodiment, which is regarded as independently inventive, the plug 18 is designed for a particularly high sealing effect within the hollow body 14. According to one aspect of this disclosure, the relatively good deformability of the comparatively soft sealing jacket 24 is specifically combined with a suitable choice of geometry and shape. For this purpose, the sealing jacket 24 has an essentially cylindrical outer jacket 40, which is provided with a plurality of circumferential sealing ribs 42 positioned at a distance from one another as seen in the direction of the cylinder axis. In the region of the sealing ribs 42, the plug 18 can be designed with a certain oversize according to one aspect of this disclosure, i.e., have a slightly larger diameter than the inner diameter of the hollow body 14. Due to the deformability of the plastic forming the sealing jacket 24, the latter can thus adapt to the inner contour of the hollow body 14, whereby, due to the shaping and the sealing ribs 42 provided, which are arranged at a distance from one another, evasive spaces are provided into which the deformed sealing ribs 42 can escape.

Thus, on the one hand, it is possible to achieve a high sealing effect in a reliable manner, while on the other hand the static and dynamic frictional forces occurring as a result of the contact with the inner wall of the respective hollow body 14 can be kept sufficiently low. According to one aspect of this disclosure, the width of the or each sealing rib 42 is selected to be smaller than the distance between two adjacent sealing ribs 42. This configuration of the stopper 18 thus makes it possible to achieve particularly good and trouble-free displaceability of the stopper 18 in the hollow body 14 while maintaining a high sealing effect.

These effects are additionally favored by the positioning and design of the retaining groove 32 with corresponding inner lip 34 provided in accordance with one aspect of this disclosure, each of which has a type of V-shaped contour when viewed in section. This creates a deformation zone 44 utilizing the deformability of the relatively soft material of the sealing jacket 24, into which material from the foremost sealing rib 42 seen in the direction of advance of the stopper 18 can escape. This further reduces the frictional resistance of the stopper 18 sliding in the syringe barrel 2 in accordance with one aspect of this disclosure.

To further favor the frictional behavior of the plug 18, according to one aspect of this disclosure, the sealing jacket 24 in the region of the end flange 30 and its inner edge 38 has a slightly enlarged inner diameter compared to the outer diameter of the central plug body 22, so that in the assembled state a gap in the manner of a circumferential groove 46 is formed at the end between these components. This circumferential groove 46 provides a further deformation zone into which material of the last sealing rib 42 seen in the direction of advance of the stopper 18 can escape in the event of a frictional load.

According to one aspect of this disclosure, the stopper 18 is produced by a 2K injection molding process.

At its distal end 4, the hollow body 14, as can be seen from the illustrations in FIGS. 3, 4, is provided with a connection piece 50 for attaching the needle head 6. In accordance with an aspect of this disclosure which is regarded as independently inventive, the connecting piece 50 has an inner channel 52 which, starting from the interior of the hollow body 14, widens continuously in the direction of its end face 54; in particular, the inner channel 52 is provided with a conical inner surface in the embodiment example. According to an aspect regarded as independently inventive, such a configuration of the connection area for the needle head 6 is particularly advantageous for the ampoule 2 intended for a pen injector, in particular in synergistic combination with the stopper 18 described above, since the combination of these elements enables an innovative, particularly efficient filling concept for filling a large number of such ampoules 2, also on an industrial scale. In such a filling concept, the ampoule 2 can be filled with the active ingredient from “above”, i.e., via the distal end 4, with its proximal end 8 already sealed with the stopper 18. The design of the inner channel 52 as a channel widening towards the end face 54 enables the temporary attachment of a correspondingly shaped filling nozzle, which can be inserted into the inner channel 52 and is securely guided therein due to its shape.

On the outside, the connecting piece 50 is provided with a circumferential outer bead 56, which forms a latching mechanism for the latching or snap-in connection provided in accordance with one aspect of this disclosure for attaching the needle head 6. Depending on the intended use, the needle head 6 can be designed in a “single-dose” injector 1, i.e., for the delivery of only one dose, or in a “multi-dose” injector 1, i.e., for the delivery of several doses.

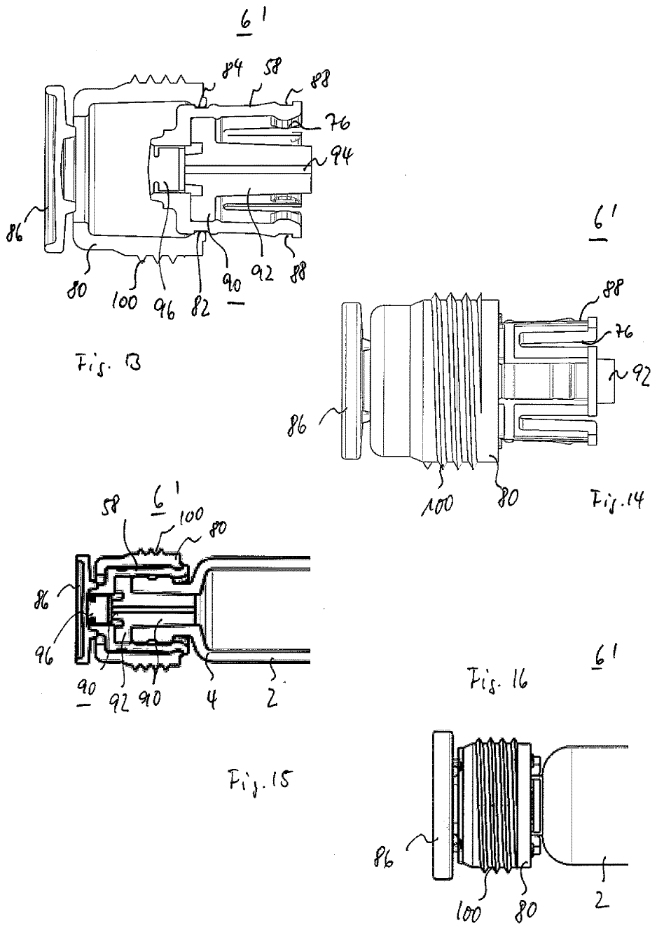

In a first variant, which can be provided in particular for a “single dose” application, and which is shown in longitudinal section in FIG. 9 and in side view in FIG. 10, the needle head 6 comprises an inner cap 58, which is designed as a two-component or 2K component in an embodiment which is regarded as independently inventive. The inner cap 58 comprises a central sealing body 60 made of a comparatively soft, sealing plastic, in the embodiment example TPE. The outer contour of the sealing body 60 is designed in the manner of a plug 62 and is adapted to the inner contour of the inner channel 52. It can thus be increasingly inserted into the inner channel 52 until, due to the conical design of the plug-in pin 62 on the one hand and the inner channel 52 adapted thereto on the other hand, a planar and thus particularly sealing contact is created between these components. Thus, according to one aspect of this disclosure, a particularly tight and, due to its shape, also mechanically particularly stable connection of the needle head 6 as a whole to the hollow body 14 is achieved. On the end side of the plug-in pin 62, the sealing body 60 has an end plate 64 designed in the form of a septum or a pierceable membrane. In an embodiment which is regarded as independently inventive, the plug-in pin 62 is designed in comparison to the inner channel 52 in such a way that the end plate 64 is positioned in the assembled state close to the inner end opening 65 of the inner channel 52, in which the latter opens into the syringe channel 16 within the hollow body 14. This is intended in particular to ensure that the stopper 18 guided in the syringe channel 16 comes completely or at least almost into contact with the end plate 64 during complete movement towards the distal end 4 of the ampoule 2, so that the dead volume after complete actuation of the stopper 18 can be minimized or at least kept particularly small. For this purpose, the end plate 64 is positioned according to one aspect of this disclosure at a distance of at most 25% of the total length of the inner channel 52 from its end opening 65.

According to one aspect of this disclosure, the sealing body 60 is arranged in a bulging cap 66 provided as a second component of the inner cap 58. The bulging cap 66 comprises an annular cover 70 having a central opening 68. On the other hand, the sealing body 60, which is designed in one piece per se, comprises at its “upper” end region opposite the end plate 64 an integrally formed outer bead 72, which is brought into abutment with the annular cover 70. A number of snap-in hooks or latching elements 74 are arranged on the ring cover 70 of the bulging cap 66, each of which can be brought into engagement with the outer bead 56 of the connection piece 50 via a latching rib 76 formed on the inside. When attaching the inner cap 58 comprising the bulging cap 66 to the connecting piece 50, it can thus be pushed or pressed onto the connecting piece 50, whereby the latching elements 74 are first bent outwards through the outer bead 56 and then, after being pushed on further, engage behind the outer bead 56 and latch with it in the manner of a snap-on connection. Such a push-on system is particularly easy to install and therefore preferred; alternatively, however, a screw connection of the inner cap 58 to the connecting piece 50 or another suitable connection could also be provided.

As a further component, as again can be seen from the illustration in FIGS. 9, 10, the needle head 6 comprises, in an embodiment which is regarded as independently inventive, an outer cap 80 which can be slid onto the bulging cap 66 of the inner cap 58. After bulging of the bulging cap 66 and latching with the outer bead 56, this outer cap can be slid onto the bulging cap 66 from the outside in an embracing manner. This fixes the latching elements 74 radially so that they can no longer move outwards. As a result, the latching of the bulge cap 66 with the outer bead 56 can no longer be easily released and is therefore fixed.

The inner cap 58 is provided on the outside with a circumferential groove 82 at the upper edge of the bulging cap 66 and in the region of the ring cover 70. Thus, according to one aspect of this disclosure, the outer cap 80 can be fitted onto the inner cap 58 in the manner of a pre-assembly, with a number of latching lugs 84 arranged on the inside of the outer cap 80 engaging in the groove 82. The needle head 6 thus forms the ensemble shown in longitudinal section in FIG. 9 and in side view in FIG. 10, which can be fitted together onto the hollow body 14.

Furthermore, according to one aspect of this disclosure, the needle head 6 is designed as a tamper-evident closure. For this purpose, it comprises a seal plate 86 formed on the outer cap 80 in the manner of a tear-off seal. When the injection device 1 is used for the first time, the seal plate 86 is torn off the outer cap 80 and the opening 68 in the inner cap 58 underneath is made accessible. This clearly indicates to the user that the injection device 1 has already been used and that the active ingredient container has been “opened”.

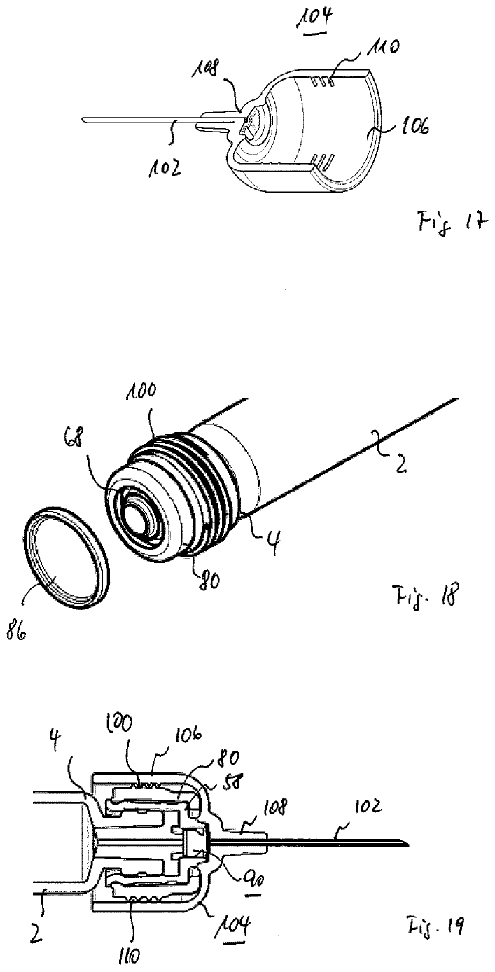

When attaching the needle head 6, the inner cap 58 can thus be pushed onto the connection piece 50 of the hollow body 14 as mentioned, whereby the latching elements 74 latch in the manner of a snap connection with the outer bead 56. For the final attachment of the needle head 6 and also for closing the distal end 4 of the ampoule 2, the outer cap 80 can then be pushed even further onto the inner cap 58, as can be seen from the representation in longitudinal section according to FIG. 11 and the representation in lateral view according to FIG. 12. According to an aspect which is considered to be independently inventive, the latching elements 74 forming the lower or skirt region of the bulging cap 66 are also each provided with a further groove 88 arranged on the outside, into which the latching lugs 84 arranged on the inside of the outer cap 80 can engage when the outer cap 80 is fully pushed on. According to one aspect of this disclosure, the outer cap 80 is thus pushed onto the inner cap 58 until the latching lugs 84 now engage in the further groove 88. The outer cap 80 is thus fixed in a latching manner on the inner cap 58 of the needle head 6, and the outer cap 80 encloses the skirt region of the bulging cap 66 in the manner of a retaining ring. This radially fixes the latching connection of the latching elements 74 engaging behind the outer bead 56, as these can now no longer move outwards and the latching is now fixed.

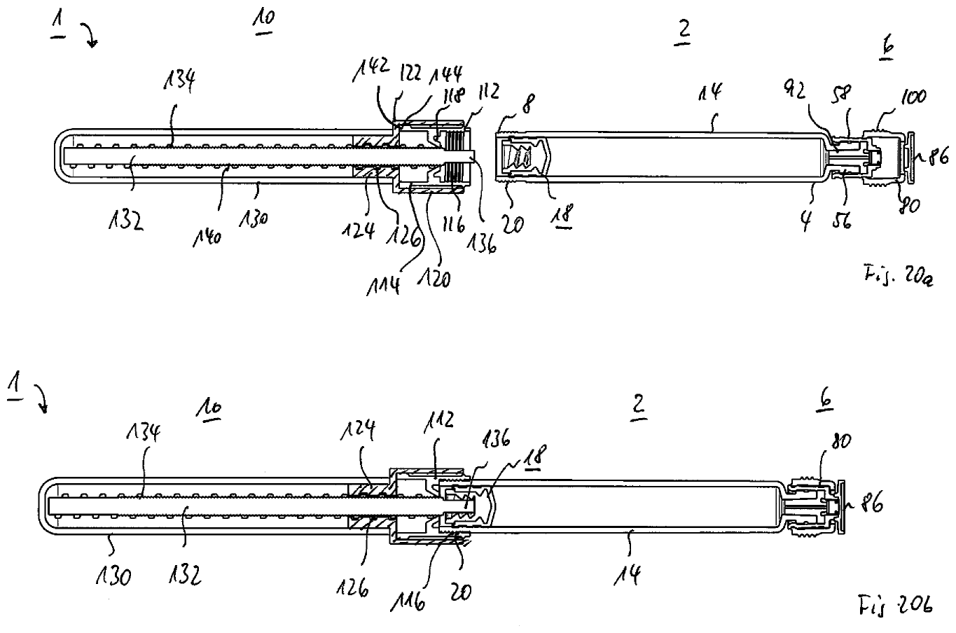

An alternative embodiment or variant of the needle head 6′, which is considered to be independently inventive and which can be provided in particular for a “multi-dose” application or the multiple delivery of individual doses of active ingredient from the injection device 1, is shown in FIG. 13 in longitudinal section and in FIG. 14 in lateral view. In this embodiment, a multi-component piston valve 90 is arranged inside the inner cap 58 of the needle head 6′ instead of the sealing body 60, with otherwise essentially the same design and functionality of the components described above. On the one hand, this comprises a channel body 92 whose outer contour is also designed, as described above, in the manner of a plug-in pin and which is adapted to the inner contour of the inner channel 52. This can thus, similarly to the plug-in pin 62 described above, be increasingly inserted into the inner channel 52 until, due to the conical design of the channel body 92 on the one hand and the inner channel 52 adapted thereto on the other hand, a planar and thus particularly sealing contact is created between these components. Thus, according to one aspect of this disclosure, a particularly tight and, due to its shape, also mechanically particularly stable connection of the needle head 6 as a whole to the hollow body 14 is also achieved in this embodiment. A media channel 94 is arranged within the channel body 92, via which medium can be conveyed from the interior of the ampoule 2 to the outside.

In addition, the piston valve 90 comprises a valve piston 96, which can also be designed as a 2C component and in particular be designed for an automatic valve function, in which a valve diaphragm arranged on the outlet side is lifted or brought into an open position when the upstream media channel is pressurized, i.e., when the medium is supplied from the interior of the ampoule 2, so that the corresponding valve channels are exposed and the medium can flow out.

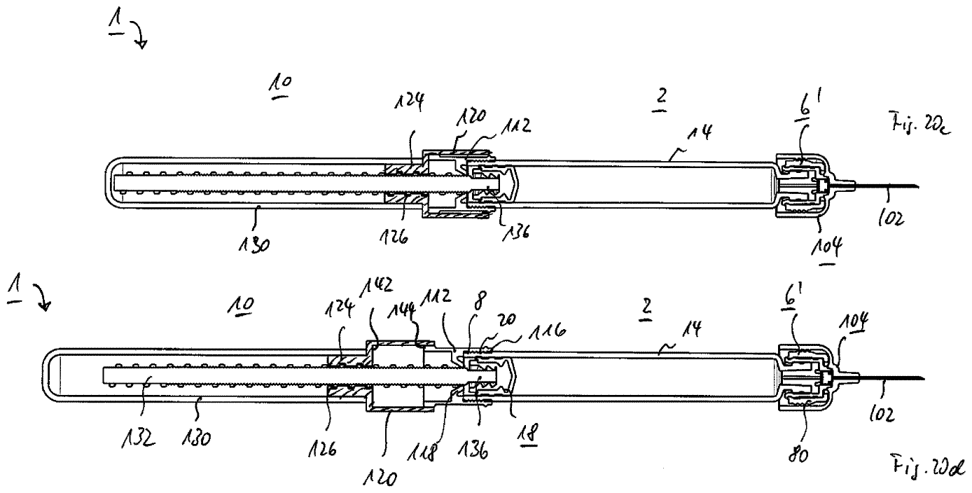

The attachment of the needle head 6′ to the ampoule 2 by snapping it on or by a snap-in connection is otherwise identical to that of the needle head 6 described above. In this embodiment, too, the outer cap 80 is pushed onto the inner cap 58 until it is finally latched and fixed in place for the final attachment of the needle head 6′ and also for closing the distal end 4 of the ampoule 2, as can be seen from the longitudinal sectional view shown in FIG. 15 and the side view shown in FIG. 16.

As can be seen from the representations of the figures described above, the outer cap 80 of the needle head 6, 6′ is provided with an external thread 100 on its outer casing in accordance with one aspect of this disclosure. In an embodiment which is considered to be independently inventive, this is provided for attaching a needle attachment 104 carrying a hollow needle 102, in particular a standard pen attachment. Before and according to one aspect of this disclosure, the external thread 100 is adapted to the corresponding parameters of conventional and standardly used pen attachments with regard to its geometry and its thread parameters, for example diameter and/or thread pitch. In summary, the needle head 6, 6′, which is considered to be independently inventive, thus comprises, in the assembled state, an inner cap 58 comprising a sealing body 60 or a piston valve 90, which is fastened in a latching manner to the distal end 4 of the ampoule 2 via the bulging cap 66, an outer cap 80 fixing the bulging cap 66 being provided on the outside, i.e., in particular in the jacket region surrounding the bulging cap 66, with an external thread 100 for attaching the needle attachment 104.

The needle attachment 104 is shown in FIG. 17 in perspective sectional view. As a base body, it comprises a cap 106, which comprises an integrally formed needle holder 108. On its inner surface, the cap 106 is provided with an internal thread 110, which is adapted to the external thread 100 in terms of its geometry and thread parameters and can therefore be screwed onto the latter.

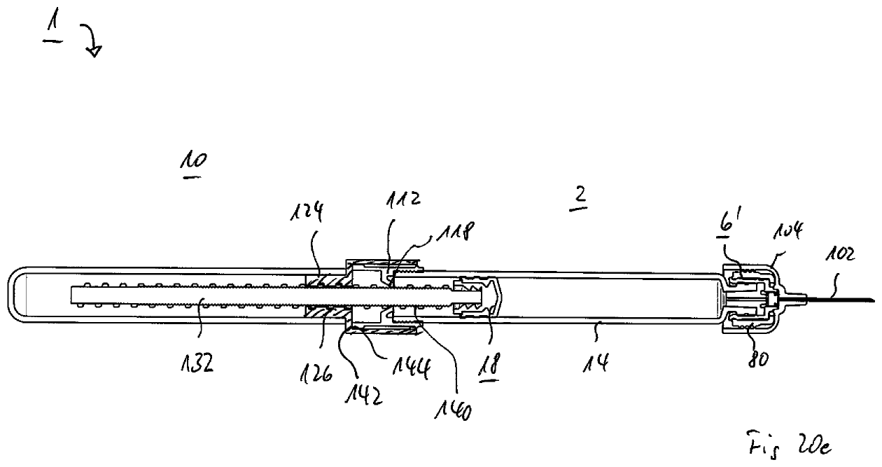

In order to make the injection device 1 ready for use, the sealing plate 86 is first removed from the needle head 6, 6′ so that the sealing element 60 or piston valve 90 underneath is exposed and thus made accessible. This is shown in FIG. 18 using the distal end 4 of the ampoule 2 shown in section. The needle attachment 104 can then be screwed on, as shown as an example for the needle head 6′ in longitudinal section in FIG. 19.

The use and application of the injection device 1, especially in the variant as a “multi-dose” device for administering a dose of active ingredient, is shown in FIG. 20 via a sequence of illustrations, each in longitudinal section. The ampoule 2 filled with the active ingredient, for example with insulin, and sealed with the stopper 18 on the one hand and with the needle head 6, 6′ on the other hand, is first (FIG. 20a) connected to the actuating element 10 by screwing it onto the external thread 20. For its part, the actuating element 10 is multi-part and is designed as an actuating and dosing element according to the following description in an embodiment which is regarded as independently inventive. It comprises a union nut 112, which is essentially formed by a hollow jacket cylinder 114. This is provided on the inside with a distal internal thread 116, the parameters of which are adapted to the external thread 20 of the ampoule 2, so that the union nut 112 can be screwed onto the proximal end 8 of the ampoule 2. Furthermore, a number of latching springs 118 are arranged approximately centrally in the jacket cylinder 114.

The sleeve cylinder 114 of the union nut 112 is axially displaceably guided in a tubular piece 120 of a rotary nut 122. The rotating nut 122 has a spindle body 124 with a spindle or steep thread 126 arranged on the inside, and it is arranged on an outer housing 130. A piston rod 132 is arranged inside the elongated outer housing 130, which forms a grip area on the outside for the user, as the main dosing mechanism for presetting the quantity of active substance to be dispensed. The piston rod 132 is provided on its outer side with a steep external thread 134 adapted to the steep thread 126 and is guided with this through the spindle body 124. At the end, the piston rod 132 opens into a connecting pin 136 which can be inserted into the stopper 18 of the injection device 1. Furthermore, the piston rod 132 is provided on the outside with a detent fine thread 140 which is superimposed on the external steep thread 134.

In a first step, the actuating element 10 is attached to the proximal end 8 of the ampoule 2 by screwing the internal thread 116 of the union nut 112 onto the external thread 20 of the ampoule. In the process, the connecting pin 136 of the piston rod 132 penetrates into the receiving channel 26 of the stopper 18 guided in the ampoule 2 and thus establishes a connection between the piston rod 132 and the stopper 18. The sealing plate 86 is then torn off from the unit shown in FIG. 20b, which is connected in this way, and the needle attachment 104 is screwed on. In this state, shown in FIG. 20c, the injection device 1 is now ready for use.

To set the predetermined dose or delivery quantity, the outer housing 130 including the spindle body 126 fixed therein is then rotated by a predetermined angle of rotation, for example limited by a suitably placed stop, relative to the ampoule 2 and the union nut 112 fixed thereto. As a result, the spindle body 126 also rotates relative to the piston rod 132, which is non-rotatably connected to the union nut 112. The rotation by a predetermined angle of rotation is thus converted by the spindle drive formed by the pairing of the steep thread 126 and the steep external thread 134 into a defined axial offset or stroke of the outer housing 130 relative to the union nut 112, which is fixedly connected to the ampoule 2. This offset can be clearly seen in the illustration in FIG. 20d, compared to the previous state in FIG. 20c.

The dose of medication can then be dispensed by pressing the outer housing 130. In the process, the outer housing 130 and with it, due to the mounting of the piston rod 132 in the spindle body 126, the piston rod 132 and the stopper 18 attached to its end are moved towards the distal end 4 of the ampoule 2, whereby the active ingredient is dispensed through the hollow needle 102. The dispensing takes place until the spindle body 126 strikes the upper edge 144 of the union nut 112 with its underside forming a stop surface 142. This limits the amount of active ingredient dispensed to the maximum amount specified by the thread parameters and angle of twist; precise dosing is made possible. When the piston rod 132 is moved through the union nut 112, the fine detent thread 140 slides through the detent springs 118 arranged in the union nut 112, which prevent a “backward movement” of the piston rod 132 due to the detent effect. The injection device 1 after the delivery of such a dose is shown in FIG. 20e. The relative offset of the piston rod 132 within the outer housing 130 is clearly recognizable. The injection device 1 is now ready to deliver the next dose of active ingredient. The delivery can be repeated until the stopper 18 in the area of the distal end 4 of the ampoule 2 has struck the end surface or the end plate 64 of the needle head 6, 6′; the ampoule 2 is then completely emptied and the doses are used up.

### LIST OF REFERENCE SYMBOLS

1 Injection device
2 Ampoule
4 distal end
6 Needle head
8 Proximal end
10 Actuating element
14 Hollow body
16 Syringe channel
18 Stopper
20 External thread
22 Plug body
24 Sealing jacket
26 Recording channel
28 Connection area
30 Flange
32 Holding groove
34 Inner lip
36 Edge
38 Inner edge
40 Outer sheath
42 Sealing rib
44 Deformation zone
46 Groove
50 Connection piece
52 Inner duct
54 Front face
56 Outer bead
58 Inner cap
60 Sealing body
62 Plug-in spigot
64 End plate
65 End opening
66 Impact cap
68 Opening
70 Ring cover
72 Outer bead
74 Latching element
76 Locking rib
80 Outer cap
82 Groove
84 Latching lug
86 Sealing plate
88 Groove
90 Piston valve
92 Channel body
94 Media channel
96 Valve piston
100 External thread
102 Hollow needle
104 Needle attachment
106 Cap
108 Needle holder
110 Internal thread
112 Union nut
114 Shell cylinder
116 Internal thread
118 Detent springs
120 Pipe section
122 Swivel nut
124 Spindle body
126 Steep thread
130 Outer housing
132 Piston rod
134 Steep external thread
136 Connecting spigot
140 Detent fine pitch thread
142 Stop surface
144 Upper edge

1. An injection device, comprising:
an active substance ampoule that is closed at a distal end by a sealing body of a needle head;
an inner cap which can be slid onto a connecting piece of the active substance ampoule;
a number of latching elements in a region designed as a bulging cap; and
an outer cap which can be slid onto the inner cap and which, when slid onto the inner cap, radially fixes the latching elements.

2. The injection device according to claim 1, wherein the inner cap of the needle head comprises a sealing body made of a softer material compared to the bulging cap.

3. The injection device according to claim 1, wherein the outer cap of the needle head is provided with an external thread on an outer casing.

4. (canceled)

5. The injection device according to claim 1, wherein the active substance ampoule is prefilled with an active substance to be administered.

6. The injection device according to claim 1, wherein the injection device is a pen injector.

7. The injection device according to claim 2, wherein the sealing body is made of TPE.

8. The injection device according to claim 2, wherein the outer cap of the needle head is provided with an external thread on an outer casing.

9. The injection device according to claim 2, wherein the active substance ampoule is prefilled with an active substance to be administered.

10. The injection device according to claim 3, wherein the active substance ampoule is prefilled with an active substance to be administered.

11. The injection device according to claim 2, wherein the injection device is a pen injector.

12. The injection device according to claim 3, wherein the injection device is a pen injector.

13. The injection device according to claim 5, wherein the injection device is a pen injector.
